(19) United States
(12) Patent Application Publication
(10) Pub. No.: US 2026/0272699 A1
(43) Pub. Date: Sep. 17, 2026

(54) Post Shower Stoma Wrap

(71) Applicant: Sharon Leann Anderson, Escondido, CA (US)

(72) Inventor: Sharon Leann Anderson, Escondido, CA (US)

(21) Appl. No.: 19/080,619

(22) Filed: Mar. 14, 2025

(51) Int. Cl. A61F 5/44

(52) U.S. Cl. CPC A61F 5/4407

(57) ABSTRACT

The Post Shower Ostomy Wrap is a specialized garment designed for individuals who rely on an ostomy or urostomy bag. This innovative wrap features an elastic band for a secure and adjustable fit, along with an absorbent pouch that holds a sterile, disposable pad to capture urine leakage from an exposed stoma. The pouch includes a mesh inner layer for fluid absorption and a water-resistant outer layer to prevent leakage. A fastening mechanism, such as Velcro or snaps, allows for easy application and size adjustments. This wrap enables users to bathe without an ostomy bag while maintaining cleanliness and preventing urine from running down the body. It provides comfort, convenience, and improved hygiene, allowing users time to dry off, groom, and dress before reapplying their ostomy bag. The Post Shower Ostomy Wrap is reusable, machine washable, and designed to accommodate various sizes and user preferences.

### BACKGROUND OF INVENTION

The inspiration for the Post Shower Stoma Wrap came from a deeply personal experience. When my mom underwent surgery to have her bladder removed due to cancer, she faced a new set of daily challenges—one of which was maintaining optimal cleanliness while showering. She wanted the freedom to remove her ostomy bag in the shower, ensuring a thorough cleanse.

However, the real issue arose once she stepped out. She needed time to dry off, brush her hair, and get dressed before reapplying the ostomy bag, but there was no effective way to keep her stoma protected in the meantime.

Through numerous conversations and brainstorming sessions with my mom, we worked together to develop a solution that would provide comfort, security, and convenience after showering.

This collaboration led to the creation of the Post Shower Stoma Wrap—a simple yet innovative product designed to offer individuals with ostomies the confidence and ease they deserve in their daily routines.

### SUMMARY OF INVENTION

The Post Shower Ostomy Wrap is an innovative solution designed for individuals who rely on an ostomy or urostomy bag. This wrap features an elastic band with an adjustable pouch that holds highly absorbent, sterile disposable pads to capture urine leakage from an exposed stoma. By preventing urine from running down the body after showering, the wrap allows users to dry off, clean up, and get dressed comfortably before reapplying their ostomy bag. The pouch incorporates a water-resistant plastic fabric on the opposite side of the absorbent mesh, ensuring cleanliness and preventing leakage. This product provides a practical and hygienic way for ostomy patients to shower without their bag while maintaining confidence and convenience in their daily routine.

### DESCRIPTION OF VIEWS OF THE DRAWINGS

FIG. 1—A detailed drawing illustrating the construction of the Post Shower Ostomy Wrap, including its components and how they are assembled.

FIG. 2—A technical drawing showcasing the various available sizes, including child and adult options, as well as the placement of the Velcro fasteners and the pouch.

FIG. 3—A black and white photograph displaying the interior of the pouch and wrap, highlighting the functionality of a working prototype.

FIG. 4—A black and white photograph showing the back view of the working prototype, providing a clear look at the materials and design structure.

FIG. 5—A black and white photograph featuring a larger-sized version of the working prototype, demonstrating its adaptability for different users.

FIG. 6—A black and white photograph focusing on the front side of the pouch, showcasing the disposable, non-adhesive absorbent pad designed for effective moisture control.

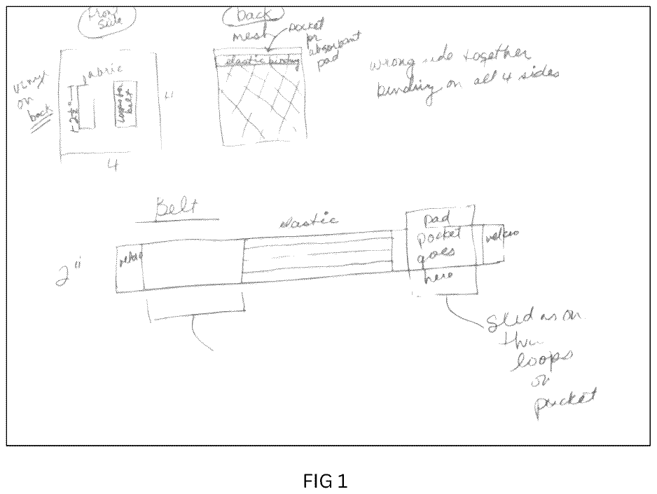

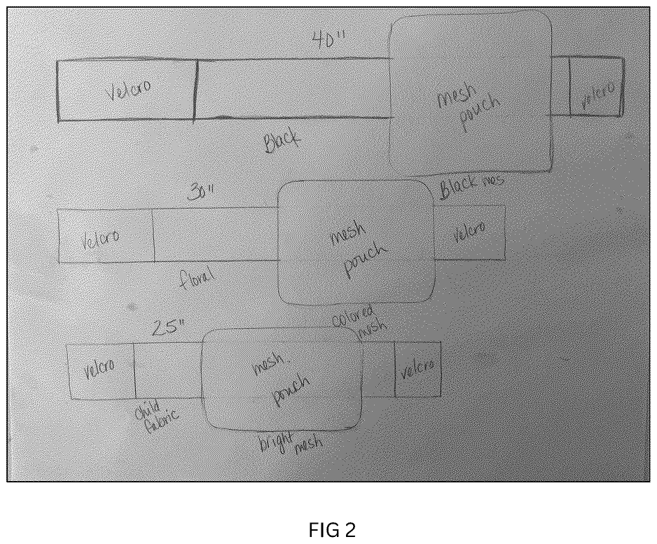

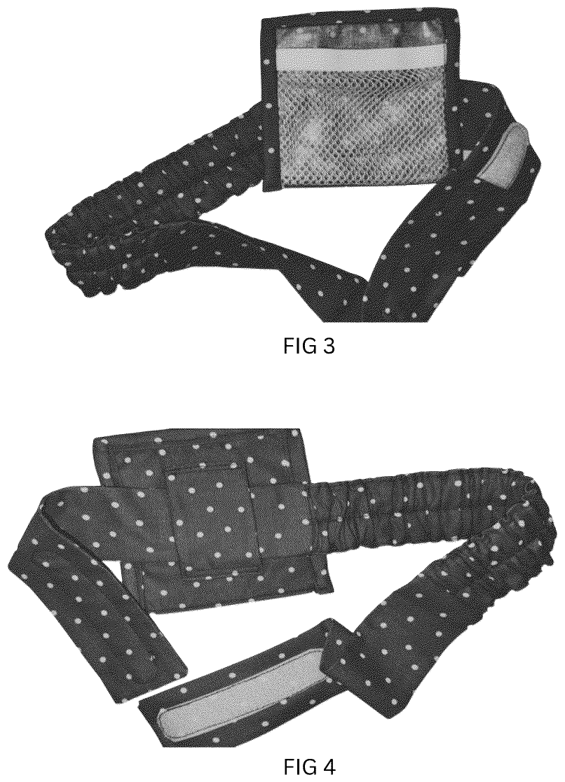

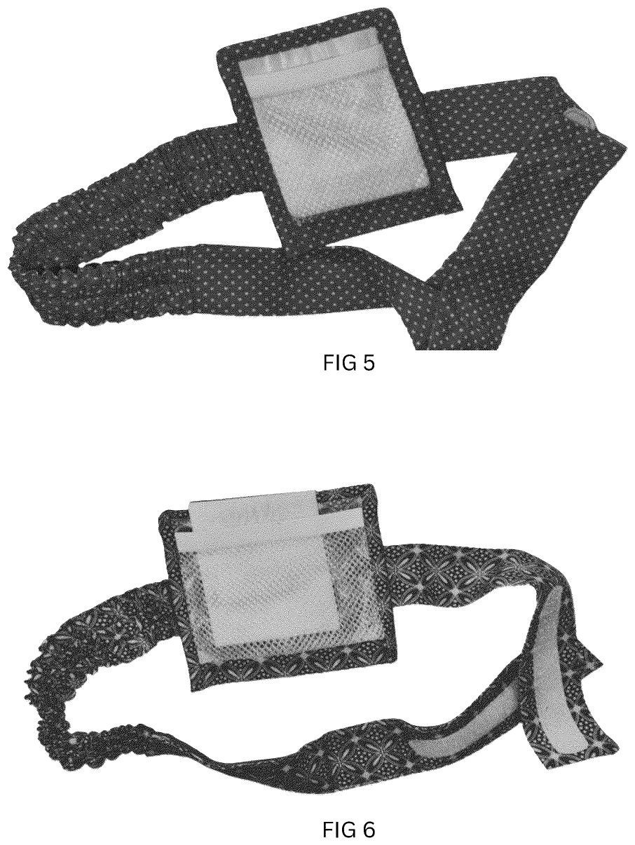

### DETAILED DESCRIPTION OF THE INVENTION

The Post Shower Ostomy Wrap is a specialized medical accessory designed to assist individuals who rely on an ostomy or urostomy bag. This innovative wrap provides a hygienic and practical solution for managing stoma output immediately after showering, allowing users to maintain cleanliness and comfort while drying off, grooming, and dressing before reapplying their ostomy bag.

The wrap consists of an elastic band that conforms to the user's body, ensuring a snug yet comfortable fit. The band features an adjustable pouch, strategically positioned to cover the stoma and collect any urine or moisture that may be expelled while the user is without their ostomy bag.

Inside the pouch, the wrap holds highly absorbent, sterile disposable pads designed to capture and contain urine leakage effectively. These pads prevent liquid from running down the user's body, maintaining dryness and preventing discomfort after showering.

To enhance its functionality, the pouch features a dual-layered fabric construction:
The inner layer consists of a breathable mesh fabric that allows for efficient absorption and retention of moisture.
The outer layer is made from a water-resistant plastic fabric, preventing leakage from soaking through to the exterior of the wrap.

### Functionality and Benefits

Prevents Urine Leakage—The absorbent pad inside the pouch captures and holds any urine output from the stoma, allowing the user to remain clean and dry.
Facilitates Post—Shower Routine-Users can comfortably take their time drying off, brushing their hair, or dressing without the urgency of immediately reapplying their ostomy bag.
Adjustable and Secure Fit—The elastic wrap ensures a customizable fit for different body sizes, making it suitable for both adults and children.
Hygienic and Disposable—The use of sterile, disposable absorbent pads promotes optimal hygiene and ease of use.
Water-Resistant Protection—The outer plastic layer prevents leaks, ensuring that urine does not soak through the pouch and onto clothing or the body.
Enhanced Comfort and Confidence—Users can bathe without their ostomy bag while maintaining dignity, independence, and peace of mind.

1. A Post Shower Ostomy Wrap designed to aid individuals who rely on an ostomy or urostomy bag, comprising an elastic band configured to secure the wrap around the user's body, ensuring a comfortable and adjustable fit. The wrap includes an adjustable pouch positioned to cover an exposed stoma, with a mesh inner layer adapted to hold a highly absorbent, sterile disposable pad designed to capture and retain urine leakage, and a water-resistant outer layer composed of plastic or similar material to prevent leakage from penetrating through to the exterior of the wrap. The fastening mechanism, including but not limited to Velcro, snaps, or equivalent securing features, allows for adjustable sizing and secure placement. The structure of the wrap is designed to enable a user to bathe without wearing an ostomy bag while preventing urine from running down the body, thereby allowing the user to dry off, groom, and dress before reapplying their ostomy bag.
The Post Shower Ostomy Wrap may include an elastic band made of a flexible, moisture-resistant material to enhance comfort and durability. The absorbent pad is non-adhesive, disposable, and replaceable for improved hygiene. The fastening mechanism allows for multiple size options, including configurations for both children and adults. The wrap may also feature reinforced stitching or sealing around the pouch to prevent leaks and ensure durability during use. The water-resistant outer layer can be composed of medical-grade plastic, vinyl, or other fluid-repellent materials. The pouch placement may be customizable based on user preference and stoma location. Additionally, the elastic band is configured for easy removal and reapplication without interfering with the absorbent pad or pouch. The wrap may further comprise a disposable pouch liner to allow for easy cleanup and replacement after each use. It is also designed to be machine washable and reusable, with the exception of the disposable absorbent pads.
This claim is intended to encompass all variations, modifications, and adaptations that fall within the scope and spirit of the Post Shower Ostomy Wrap as described herein.
